(19) United States
(12) Patent Application Publication
(10) Pub. No.: US 2015/0351668 A1
(43) Pub. Date: Dec. 10, 2015

(54) METHOD FOR DETECTION OF LATENT FINGERPRINTS APPLIED POSTMORTEM TO A CORPSE

(71) Applicant: Deborah M. Wichlep, Boerne, TX (US)

(72) Inventor: Deborah M. Wichlep, Boerne, TX (US)

(21) Appl. No.: 14/299,454

(22) Filed: Jun. 9, 2014

(51) Int. Cl. A61B 5/117; G06K 9/00

(52) U.S. Cl. CPC A61B 5/1172; G06K 9/00033

(57) ABSTRACT

A method for detection of latent fingerprints applied postmortem to a corpse includes the steps of providing a print developing solution, spraying the print developing solution to a portion of the corpse suspected of harboring a postmortem applied latent fingerprint, and photographing the portion of the corpse to record a detected fingerprint. The print developing solution is a mixture of water, molybdenum disulfide, one or more surfactants and one or more solvents. The surfactant may include sodium laureth sulfate, ammonium lauryl sulfate or a combination thereof. The solvent may include glycerin, polyethylene glycol or a combination thereof. In at least one formulation, both the surfactant and the solvent may be obtained from commercially available liquid hand soap. The corpse may be processed for latent fingerprints applied postmortem whether, at the time of processing, the corpse is wet or dry.

### FIELD OF THE INVENTION

The present invention relates to forensic science. More particularly, the invention relates to a solution and related method for detection of latent fingerprints applied postmortem to a corpse.

### BACKGROUND OF THE INVENTION

In the art of forensic fingerprinting, a fingerprint applied postmortem to a corpse can be the proverbial “smoking gun.” In particular, because in general fingerprints cannot be applied to a body until some period of time after death, the discovery of a fingerprint on a corpse not only potentially identifies the actor, but is also generally indicative of his or her evil intent. To be sure, in homicide cases, such fingerprints are generally left by actors attempting to hide the corpse or manipulate the crime scene.

Unfortunately, collection of such latent fingerprints applied postmortem to a corpse is notoriously difficult and always time consuming, each of the previously known methods suffering significant limitations. For example, direct transfer by glass plate requires that the plate be pressed onto the skin of the cadaver and then processed with magnetic powder. In order to be effective at all, however, an evidence technician must generally have some a priori knowledge of where a latent print is.

By way of further example, widely used cyanoacrylate (superglue) techniques to polymerize fingerprints require portable chambers and volatile fuming kits, are tedious and extremely time consuming and, of much concern, are often destructive of other trace evidence, especially hairs, fibers and residues. What is more, such techniques can only be applied if the corpse is dry. Even then, however, the techniques generally produce suboptimal results. In particular, with normal cyanoacrylate fuming the resulting prints are difficult to observe on opaque surfaces, especially white surfaces, and with cyanoacrylate fuming followed by application of a staining agent, absorption of dye into the skin considerably reduces the contrast between the print and background, resulting in loss of ridge detail.

With these and other known limitations of the prior art clearly in mind, it is therefore an overriding object of the present invention to provide a simple and effective method for detection of latent fingerprints applied postmortem to a corpse, whether at the time of detection the corpse is wet or dry or warm or cold.

Additionally, it is an object of the present invention to provide such a method for detection of latent fingerprints applied postmortem to a corpse that is fast to conduct, preferably requiring only seconds to fully accomplish. Still further, it is an object of the present invention to provide such a method for detection of latent fingerprints applied postmortem to a corpse that is cost effective, relying only on a simple solution and requiring no complex, expensive or non-reusable equipment, such as fuming tents, specialized lights or cameras and the like.

Finally, it is an object of the present invention to provide such a method for detection of latent fingerprints applied postmortem to a corpse that is generally safe for use by the evidence technician, requiring no volatile or hazardous chemicals.

### SUMMARY OF THE INVENTION

In accordance with the foregoing objects, the present invention—a method for detection of latent fingerprints applied postmortem to a corpse—generally comprises:

providing a print developing solution, said print developing solution comprising water; molybdenum disulfide; a surfactant; and a solvent;

applying said print developing solution to a portion of the corpse suspected of harboring a postmortem applied latent fingerprint; and imaging said portion of the corpse to record a detected fingerprint.

The surfactant may comprise sodium laureth sulfate, ammonium lauryl sulfate or a combination of sodium laureth sulfate and ammonium lauryl sulfate. The solvent may comprise glycerin or polyethylene glycol and, most preferably, comprises a combination of both glycerin and polyethylene glycol. To this end, the surfactant(s) and solvent(s) may be conveniently obtained by utilization to this end of a commercially available liquid hand soap comprising sodium laureth sulfate, ammonium lauryl sulfate, glycerin and polyethylene glycol.

The corpse may be processed for latent fingerprints applied postmortem whether, at the time of processing, the corpse is wet or dry. The imaging may be readily accomplished with conventional photographic means, which is conducive to documentation of revealed fingerprints along with other evidentiary tools, such as, for example, an L-shaped scale and/or identification placard.

Finally, many other features, objects and advantages of the present invention will be apparent to those of ordinary skill in the relevant arts, especially in light of the foregoing discussions and the following drawings, exemplary detailed description and appended claims.

### DETAILED DESCRIPTION OF THE PREFERRED EMBODIMENT

Although those of ordinary skill in the art will readily recognize many alternative embodiments, especially in light of the illustrations provided herein, this detailed description is exemplary of the preferred embodiment of the present invention, the scope of which is limited only by the claims appended hereto.

In accordance with the preferred implementation of the present invention, a method for detection of latent fingerprints applied postmortem to a corpse begins with the preparation or other provision of print developing solution suitable for application to a corpse, whether the corpse be wet or dry, and which, when applied will visibly reveal any latent fingerprints as may be present in the area of application. To this end, Applicant has discovered a novel developing solution comprising a mixture of: water; molybdenum disulfide; one or more surfactants, such as, for example, sodium laureth sulfate and/or ammonium lauryl sulfate; and one or more solvents, such as, for example, glycerin and/or polyethylene glycol.

Although Applicant does not wish to be tied to any particular theory of operation, it is believed that the described composition is effective due to a number of contributing factors. First, it is believed that by lowering the surface tension of the water base of the solution, the surfactant or surfactants operate to facilitate release of the molybdenum disulfide for adherence to the fats forming a latent fingerprint, whereupon the molybdenum disulfide, which is similar in appearance and feel to graphite, forms a grey colored deposit visibly revealing the ridge pattern of the previously latent fingerprint. Second, it is believed that the solvents facilitate distribution of the molybdenum disulfide, thereby ensuring uniform and complete coverage of the latent fingerprint with the suspended molybdenum disulfide.

Still further, however, it is noted that the particular solvents discovered by Applicant may serve additional useful functions in operation of the present invention. In particular, it is noted that glycerin is also a humectant, which may serve to rehydrate dried fats of a latent fingerprint, thereby making these fats receptive to the molybdenum disulfide and that polyethylene glycol is also a binder, which may serve to further enhance the consistency of the prepared or otherwise provided print developing solution.

With these discoveries in mind, Applicant has found that a suitable formulation may be prepared as follows:

### Print Developing Solution

| | | |
|---|---|---|
| Water | 1 | L |
| Molybdenum Disulfide | 25-50 | g |
| Liquid Hand Soap | 15 | mL |

where the utilized liquid hand soap comprises typical commercial quantities of sodium laureth sulfate, ammonium lauryl sulfate, glycerin and polyethylene glycol.

With the suitable print developing solution on hand, the print developing solution is preferably contained in a conventional spray bottle for application to a portion of a corpse suspected of harboring a postmortem applied latent fingerprint. The solution is sprayed onto the suspect area until the previously latent fingerprint becomes visible or, in the alternative, it is determined that no fingerprint is present. Assuming a fingerprint is present, substantially pure water may, as necessary, be separately sprayed over the same portion of the corpse to remove any excess molybdenum disulfide as may be occluding a clear ridge pattern. In any case, the discovered fingerprint is then imaged for evidentiary purposes. Because the described method produces a vividly observable ridge pattern, the imagery may be conveniently and rapidly accomplished by photographing the revealed fingerprint along with any other desired evidentiary tools, such as, for example, an L-shaped scale and/or identification placard.

As will be appreciated by those of ordinary skill in the art in light of the foregoing exemplary description, the foregoing method stands as a dramatic improvement in fingerprint forensics involving fingerprints applied postmortem to a corpse. In particular, the described method is fast, reliable and simple and, unlike many other techniques previously employed, is generally nondestructive of other trace evidence.

While the foregoing description is exemplary of the preferred embodiment of the present invention, those of ordinary skill in the relevant arts will recognize the many variations, alterations, modifications, substitutions and the like as are readily possible, especially in light of this description, the accompanying drawings and claims drawn thereto. In any case, because the scope of the present invention is much broader than any particular embodiment, the foregoing detailed description should not be construed as a limitation of the scope of the present invention, which is limited only by the claims appended hereto.

What is claimed is:

1. A method for detection of latent fingerprints applied postmortem to a corpse, said method for detection of latent fingerprints comprising the steps of:
providing a print developing solution, said print developing solution comprising:
water;
molybdenum disulfide;
a surfactant; and
a solvent;
applying said print developing solution to a portion of the corpse suspected of harboring a postmortem applied latent fingerprint; and
imaging said portion of the corpse to record a detected fingerprint.

2. The method for detection of latent fingerprints applied postmortem to a corpse as recited in claim 1, wherein said surfactant comprises sodium laureth sulfate.

3. The method for detection of latent fingerprints applied postmortem to a corpse as recited in claim 1, wherein said surfactant comprises ammonium lauryl sulfate.

4. The method for detection of latent fingerprints applied postmortem to a corpse as recited in claim 2, wherein said surfactant further comprises sodium laureth sulfate.

5. The method for detection of latent fingerprints applied postmortem to a corpse as recited in claim 1, wherein said solvent comprises glycerin.

6. The method for detection of latent fingerprints applied postmortem to a corpse as recited in claim 1, wherein said solvent comprises polyethylene glycol.

7. The method for detection of latent fingerprints applied postmortem to a corpse as recited in claim 8, wherein said solvent further comprises glycerin.

8. The method for detection of latent fingerprints applied postmortem to a corpse as recited in claim 7, wherein said surfactant comprises sodium laureth sulfate.

9. The method for detection of latent fingerprints applied postmortem to a corpse as recited in claim 7, wherein said surfactant comprises ammonium lauryl sulfate.

10. The method for detection of latent fingerprints applied postmortem to a corpse as recited in claim 9, wherein said surfactant further comprises sodium laureth sulfate.

11. The method for detection of latent fingerprints applied postmortem to a corpse as recited in claim 10, wherein said corpse is substantially dry.

12. The method for detection of latent fingerprints applied postmortem to a corpse as recited in claim 10, wherein said corpse is wet.

13. The method for detection of latent fingerprints applied postmortem to a corpse as recited in claim 12, wherein said applying said print developing solution comprises spraying said print developing solution onto said portion of the corpse suspected of harboring a postmortem applied latent fingerprint.

14. The method for detection of latent fingerprints applied postmortem to a corpse as recited in claim 13, said method for detection of latent fingerprints further comprising the step of rinsing excess molybdenum disulfide from said portion of the corpse suspected of harboring a postmortem applied latent fingerprint.

15. The method for detection of latent fingerprints applied postmortem to a corpse as recited in claim 14, wherein said rinsing excess molybdenum disulfide step comprises applying water to said portion of the corpse suspected of harboring a postmortem applied latent fingerprint.

16. The method for detection of latent fingerprints applied postmortem to a corpse as recited in claim 15, wherein said applying water step comprises spraying water onto said portion of the corpse suspected of harboring a postmortem applied latent fingerprint.

17. The method for detection of latent fingerprints applied postmortem to a corpse as recited in claim 16, wherein said imaging step comprises photographing said portion of the corpse.

18. A method for detection of latent fingerprints applied postmortem to a corpse, said method for detection of latent fingerprints comprising the steps of:
providing a print developing solution, said print developing solution comprising:
water;
molybdenum disulfide;
sodium laureth sulfate;
glycerin; and
polyethylene glycol;
spraying said print developing solution to a portion of the corpse suspected of harboring a postmortem applied latent fingerprint;
spraying water onto said portion of the corpse suspected of harboring a postmortem applied latent fingerprint, thereby rinsing excess molybdenum disulfide from said portion of the corpse suspected of harboring a postmortem applied latent fingerprint; and
photographing said portion of the corpse to record a detected fingerprint.

19. A method for detection of latent fingerprints applied postmortem to a corpse, said method for detection of latent fingerprints comprising the steps of:
providing a print developing solution, said print developing solution comprising:
water;
molybdenum disulfide;
ammonium lauryl sulfate;
glycerin; and
polyethylene glycol;
spraying said print developing solution to a portion of the corpse suspected of harboring a postmortem applied latent fingerprint;
spraying water onto said portion of the corpse suspected of harboring a postmortem applied latent fingerprint, thereby rinsing excess molybdenum disulfide from said portion of the corpse suspected of harboring a postmortem applied latent fingerprint; and
photographing said portion of the corpse to record a detected fingerprint.
